(19) United States
(12) Patent Application Publication
(10) Pub. No.: US 2005/0239133 A1
(43) Pub. Date: Oct. 27, 2005

(54) Methods to identify true antagonists and inverse agonists of the cannabinoid receptor

(75) Inventor: Peter Greasley, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca A B, Sodertalje (SE)

(21) Appl. No.: 10/521,420

(22) PCT Filed: Jul. 14, 2003
(86) PCT No.: PCT/GB03/03066

§ 371 Date: Jan. 14, 2005

(30) Foreign Application Priority Data
Jul. 17, 2002 (SE) 0202242-4

(51) Int. Cl. G01N 33/53; C07K 14/705; A61K 38/17

(52) U.S. Cl. 435/7.1; 514/2; 530/350

(57) ABSTRACT

The present invention relates to a method to identify a true antagonist and an inverse agonist of a cannabinoid receptor (CB), and to discriminate between them. The invention further relates to the use of these true antagonists and inverse agonists in the treatment of CB associated disorders such as obesity, psychiatric and neurological disorders.

### CROSS-REFERENCE TO RELATED APPLICATIONS

This application is a national stage filing under 35 U.S.C. 371 of International Application No. PCT/GB2003/003066, filed Jul. 14, 2003, which claims priority from Sweden Application No. 0202242-4, filed Jul. 17, 2002, the specification of which is incorporated by reference herein. International Application No. PCT/GB2003/003066 was published under PCT Article 21(2) in English.

### FIELD OF THE INVENTION

The present invention relates to a method to identify a true antagonist and an inverse agonist of the cannabinoid receptor. The invention further relates to the use of these true antagonists and inverse agonists in the treatment of cannabinoid receptor associated disorders such as obesity, psychiatric and neurological disorders.

### BACKGROUND OF THE INVENTION

Preparations of Cannabis sativa have been used for medicinal and recreational purposes for at least 4,000 years. Recently, cannabinoids (CB) have been the subject of renewed interest for their potential medicinal applications.

CB's exert their effects by binding to specific G-protein-coupled receptors located in the cell membrane. To date there are two known subtypes of CB receptors, CB1 and CB2. The CB1 receptor is primarily but not exclusively expressed in the central nervous system (CNS) and is believed to mediate the CNS effects of endogenous (e.g., anandamide) and exogenously applied CBs. CB2 receptor expression is however restricted to the periphery and is expressed in the spleen, tonsils and immune cells.

With an increased understanding of the biology of the CB receptor family, there has been much speculation that antagonism of CB receptors may have important therapeutic applications. For example, antagonists of the CB receptors have been speculated to be useful to treat anxiety, emesis, obesity, movement disorders, and glaucoma (Porter et al. Pharmacology & Therapeutics. 90(1):45-60, 2001), and to alleviate pain.

However, the choice of the most effective CB receptor antagonist is complicated because CB receptor antagonists can exhibit a spectrum of different antagonistic properties, for example, a CB receptor antagonist may act as a true antagonist or as an inverse agonist. It is important in the development of an effective, therapeutic CB receptor antagonist to be able to accurately functionally characterize the CB receptor antagonist. Present methods for characterizing the functionality of a CB receptor inhibitory agent are not sufficiently sensitive to allow for the easy differentiation of an antagonist from an inverse agonist. Thus, there is a need for an improved assay system whereby the functional identity of a CB receptor inhibitory agent can be accurately determined.

### SUMMARY OF THE INVENTION

The invention is directed to variant forms of cannabinoid receptors, including variant CB1 receptors and to a novel method to identify the exact functional nature of a CB inhibitory agent. The information provided by this method allows the accurate discrimination of the inhibitory agent as a true CB receptor antagonist or an inverse agonist. This will ultimately allow an agent's functionality to be correlated with the most desired in vivo therapeutic effects and will be critical for choosing a drug with the most desired properties. For example, when treating a CB associated disease it may be preferable to eliminate any CB receptor activity and, for these occasions, the choice of a CB inverse agonist will be appropriate. On other occasions, it may be preferable to maintain the intrinsic activity of the CB receptor, and therefore the choice of a CB receptor antagonist would be appropriate. The method described herein provides for the first time an easy means of characterizing a CB receptor inhibitory agent's activity and this information will ultimately be useful for the effective treatment of CB associated diseases.

In one aspect, the invention features a constitutively active CB receptor. In one embodiment, the constitutively active CB receptor is a human CB1 receptor. The CB1 receptor can comprise an alanine at position 213 of the human wild type CB1. Alternatively, the constitutively active CB1 receptor is a human CB1 receptor comprising an alanine at position 338 of the human wild type CB1. Alternatively, the constitutively active CB1 receptor is a human CB1 receptor comprising an alanine at position 213 and an alanine at position 338 of the human wild type C31.

Thus, according to one aspect of the invention there is provided an isolated nucleic acid sequence comprising a nucleotide sequence that encodes a variant cannabinoid receptor, wherein either or both of the amino acids located at position 3:49 and 6:32 (according to the system proposed by Ballesteros J A and Weinstein H (1995) Methods Neurosci 25, 366-428) is substituted for by another amino acid so as to create a constitutive vaiant form of the cannabinoid receptor.

In one embodiment the amino acid at position 3:49 and/or 6:32 is substituted for by an alanine residue.

In a further embodiment the cannabinoid receptor is CB1 or CB2.

In a further embodiment the cannabinoid receptor is CB1 wherein either or both the amino acids at positions 3:49 and 6:32 are substituted for by alanine residues.

Further aspects of the invention extend to polypeptides encoded by such nucleic acids. In a particular embodiment the invention provides an isolated CB1 receptor variant wherein either or both the amino acids at positions 3:49 and 6:32 are substituted for by alternative amino acids, and in one particular embodiment by alanine residues.

The invention also extends to host cells transformed or transfected with the nucleic acids of the invention. The transformed cells may, for example, be mammalian, bacterial, yeast or insect cells.

Included within the scope of the present invention are alleles of the constitutively active cannabinoid receptor genes and proteins of the invention, as well as variants with conservative changes and codon-optimised nucleic acids. As used herein, an “allele” or “allelic sequence” is an alternative form of a given gene. Alleles result from mutations and different alleles may encode polypeptides whose structure or function may or may not be altered. Any given gene may have one or many allelic forms. Common mutational changes, which give rise to alleles, are generally ascribed to natural deletions, additions or substitutions of nucleotides. Each of these types of changes may occur alone, or in combination with the others, one or more times in a given sequence.

In one aspect the invention features a method for identifying an inverse agonist of a CB receptor. The method includes measuring the activity of a constitutively active CB receptor; contacting a CB receptor test inhibitory agent with the constitutively active CB receptor; and measuring the activity of the constitutively active CB receptor following contact with the inhibitory agent, wherein a decrease in the activity of the constitutively active CB receptor, compared to the activity of the constitutively active CB receptor in the absence of the inhibitory agent, indicates that the agent is an inverse agonist. The constitutively active CB receptor can be a CB1 receptor, or a variant thereof, a CB2 receptor, or a variant thereof. In one embodiment, the constitutively active CB1 receptor is a human CB1 receptor comprising an alanine at position 213 of the human wild type CB1 receptor. Alternatively, the constitutively active CB1 receptor is a human CB1 receptor comprising an alanine at position 338 of the human wild type CB1. Alternatively, the constitutively active CB1 receptor is a human CB1 receptor comprising an alanine at position 213 and an alanine at position 338 of the human wild type CB1. A representative example of the sequence of this type of polypeptide is disclosed in SEQ ID NO:1.

In another aspect, the invention features a method for determining if a CB receptor inhibitory agent is an inverse agonist or a true antagonist of a CB receptor. The method includes: contacting a test CB receptor inhibitory agent with a wild-type CB receptor in the presence of a CB receptor agonist; contacting the agent with a constitutively active CB receptor and measuring the activity of the wild-type CB receptor and the constitutively active CB receptor. An inverse agonist is identified if there is a decrease in the activity in both the wild-type CB receptor and the constitutively active CB receptor. Alternatively, a true antagonist is identified if there is a decrease in the activity in the wild-type CB receptor, but not of the activity of the constitutively active CB receptor. The constitutively active CB receptor can be a CB1 receptor, or a variant thereof, a CB2 receptor, or a variant thereof, or any other member of the cannabinoid receptor family. In one embodiment, the constitutively active CB1 receptor is a human CB1 receptor comprising an alanine at position 213 of the human wild type CB 1. Alternatively, the constitutively active CB1 receptor is a human CB1 receptor comprising an alanine at position 338 of the human wild type CB1. Alternatively, the constitutively active CB1 receptor is a human CB1 receptor comprising an alanine at position 213 and an alanine at position 338 of the human wild type CB1. The wild-type CB receptor can be a CB1 receptor, or a variant thereof, a CB2 receptor, or a variant thereof. The CB agonist can be any CB agonist such as CP55940 or HU210.

The invention also features a method for identifying an inverse agonist of a CB receptor. The method includes measuring the activity of a constitutively active CB receptor expressed in a cell, e.g., a mammalian cell, an insect cell, or a yeast cell; contacting a CB receptor test inhibitory agent with the cell expressing the constitutively active CB receptor; and measuring the activity of the constitutively active CB receptor following contact with the inhibitory agent, wherein a decrease in the activity in the constitutively active CB receptor compared to the activity of the constitutively active CB receptor in the absence of the inhibitory agent indicates that the agent is an inverse agonist. The constitutively active CB receptor can be a CB1 receptor, or a variant thereof, a CB2 receptor, or a variant thereof, or any other member of the cannabinoid receptor family. In one embodiment, the constitutively active CB1 receptor is a human CB1 receptor comprising an alanine at position 213 of the human wild type CB1 receptor. Alternatively, the constitutively active CB1 receptor is a human CB1 receptor comprising an alanine at position 338 of the human wild type CB1 receptor. Alternatively, the constitutively active CB1 receptor is a human CB1 receptor comprising an alanine at position 213 and an alanine at position 338 of the human wild type CB1.

The invention further features a method for determining if a CB receptor inhibitory agent is an inverse agonist or a true antagonist of a CB receptor. The method includes identifying a test CB receptor inhibitory agent; contacting the agent with a cell, e.g., a mammalian cell, an insect cell, or a yeast cell, expressing a wild-type CB receptor in the presence of a CB agonist; contacting the agent with a cell expressing a constitutively active CB receptor; measuring the activity of the wild-type CB receptor and the constitutively active CB receptor. An inverse agonist is identified if there is a decrease in the activity in both the wild-type CB receptor and the constitutively active CB receptor. Alternatively, a true antagonist is identified if there is a decrease in the activity in the wild-type CB receptor, but not of the activity of the constitutively active CB receptor. The constitutively active CB receptor can be a CB1 receptor, or a variant thereof, a CB2 receptor, or a variant thereof, or any other member of the cannabinoid receptor family. In one embodiment, the constitutively active CB1 receptor is a human CB1 receptor comprising an alanine at position 213 of the human wild type CB1 receptor. Alternatively, the constitutively active CB1 receptor is a human CB1 receptor comprising an alanine at position 338 of the human wild type CB1 receptor. The wild-type CB receptor can be a CB1 receptor, or a variant thereof, a CB2 receptor, or a variant thereof. The CB agonist can be any CB agonist such as CP55940 or HU210.

The method also features a true antagonist or an inverse agonist identified by the method above for use as a medicament.

Also within the invention is a pharmaceutical formulation comprising a true antagonist or an inverse agonist as identified by the method above, and a pharmaceutically acceptable adjuvant, diluent or carrier.

Further the invention features use of a true antagonist or inverse agonist as identified by the method above in the preparation of a medicament for the treatment or prevention of a disorder such as obesity, associated with a CB receptor.

The invention also includes a method of treating a CB associated disorder, such as obesity, comprising administering a pharmacologically effective amount or the true antagonist or inverse agonist as identified by the method above to a patient in need thereof.

As used herein, a “constitutively active CB receptor” is a CB receptor which has been mutated to have a greater intrinsic activity compared to the wild-type CB receptor.

As used herein, “intrinsic activity” is the level of agonist independent activity at a CB receptor.

As used herein, “an inhibitory agent” or a “test inhibitory agent” is an agent that has been identified to have inhibitory effect on the activity of a CB receptor.

As used herein, “operatively linked” refers to insertion of a nucleic acid molecule into an expression vector in a manner such that the molecule can be expressed in a host cell.

As used herein, the phrases “CB receptor activity,” and “receptor activity” refer to the ability of the CB receptor to transduce a signal. The signal is transmitted through the signal transduction pathway, ultimately resulting in a cellular response. The magnitude of the cellular response can be measured to quantitate the receptor signaling activity. There are many ways of measuring CB receptor activity, such as using GTPγS assays, inhibition of cAMP production assays and reporter gene assays.

As used herein, “CB receptor” refers to CB1 and CB2 receptors and any other member of the cannabinoid receptor family. Also included are: biologically active variants thereof, such as splice variants; and biologically active portions thereof. The CB receptor, such as CB1 and CB2, can be from any animal including human, rat, mouse, and dog.

The term “substantially purified”, as used herein, refers to nucleic or amino acid sequences that are removed from their natural environment, isolated or separated, and are at least 60% free, preferably 75% free, and most preferably 90% free from other components with which they are naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density. Methods for purifying proteins are known in the art.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

### DESCRIPTION OF THE INVENTION

Similar to other G-protein-coupled receptors (GPCRs), antagonists of the CB receptor can exhibit different efficiencies and can act as “true antagonists” or “inverse agonists”. The reason for this difference in efficiencies is due to the fact that the receptor possesses a low level of intrinsic activity, i.e., an activity that occurs in the absence of an agonist. Different theories abound as to why the receptor has a low level of intrinsic activity. In one theory it is speculated that intrinsic activity results from a small percentage of total CB receptors on a cell existing, at a given time, in an active conformation and thereby initiating signal transduction even in the absence of agonists.

An agent which is a “true antagonist” is one that can inhibit the activity of an agonist-stimulated CB receptor, but can not affect the intrinsic activity of the receptor. Thus, the ability of an agent to act as a true antagonist can only be realized if the CB receptor is first agonist stimulated. The addition of a “true antagonist” would then result in the inhibition of the agonist's stimulated receptor activity.

In contrast, an agent which can act as an “inverse agonist” is one that can inhibit the intrinsic activity of the receptor. Thus, to be able to determine if an agent can act as an inverse agonist of a CB receptor it is important to be able to easily measure its spontaneous intrinsic activity. At present this is very difficult because the intrinsic activity of a wild-type CB receptor is low, thereby making the detection of an inhibitory affect on its intrinsic activity by an agent very difficult. The lack of a method for measuring the intrinsic activity precludes the classification of known ligands as antagonists or inverse agonists resulting in the ambiguous description of their properties (Barth, Expt Opin Ther Patients 8 (3) 301-314, (1999)).

The present invention provides a method to accurately determine the basal activity of a CB receptor and thereby a means of being able to accurately characterize the activity of a CB receptor inhibitory agent. In the present method, constitutively active mutants have been developed which display an increased level of intrinsic activity, thus making it possible to easily measure the intrinsic activity of a CB receptor. Thus, the present method provides a means of identifying if a test inhibitory agent is a true antagonist or an inverse agonist of a CB receptor. Since the present method provides a very sensitive method for differentiating an inverse agonist from a true antagonist, it also provides a means of discriminating whether the inverse agonist, is a partial or full inverse agonist and similarly can be used to determine if an antagonist is a partial or full antagonist.

Identifying an Inhibitory Agent of a CB Receptor

The present invention can be performed using a CB receptor inhibitory agent that has been previously identified to have antagonistic activity, or the present invention can be performed on newly identified CB receptor inhibitory agents, or as yet untested compounds.

Additional CB receptor inhibitory agents can be identified by a variety of methods known in the art such as using GTPγS assays, inhibition of cAMP production assays and reporter gene assays (all described in—Signal Transduction: A Practical Approach Edited by G. Milligan. Oxford University Press (1999).

In one screening method, a cell-based assay in which a cell which expresses a CB receptor, or biologically active portion thereof, is contacted with a test compound in the presence of a CB receptor ligand and the ability of the test compound to modulate CB receptor activity in the presence of the CB receptor ligand is determined. Determining the ability of the test compound to modulate the ability of the CB receptor to bind to a CB receptor ligand such as CB can be accomplished, for example, by coupling the CB with a radioisotope or enzymatic label such that binding of the CB to the CB receptor can be determined by detecting the labeled CB in a complex. For example, a CB receptor ligand can be labelled with ¹²⁵I, ³⁵S, ¹⁴C, or ³H, either directly or indirectly, and the radioisotope detected by direct counting of radioemmission or by scintillation counting. Alternatively, CB receptor ligand can be enzymatically labelled with, for example, horseradish peroxidase, alkaline phosphatase, or luciferase, and the enzymatic label detected by determination of conversion of an appropriate substrate to product.

In another embodiment, an assay is a cell-based assay comprising contacting a cell expressing a CB receptor. The method includes stimulating the receptor with an agonist, then adding a test compound, and determining the ability of the test compound to inhibit the activity of the CB receptor. Determining the ability of the test compound to inhibit a CB receptor can be accomplished, for example, by detecting induction of a cellular second messenger. It is known in the art that CB receptors are coupled to the transduction pathway via the G-protein Gi. Activation of the CB receptor leads to inhibition of adenylate cyclase and activation of MAP kinase. CB1 receptors can also modulate ion channels, inhibiting calcium channels, stimulating inwardly rectifying K⁺ channels and enhancing the activation of the A-type K⁺ channel. Thus, the ability of a test compound to modulate the activity of a second messenger such as adenylate cyclase, MAP kinase, or Ca²⁺ can be used to determine if the test compound is an inhibitory agent.

Any assay for measuring adenylate cyclase activity of a CB receptor can be used. For example, the generation of radiolabeled cAMP can be quantitated as a measure of adenylate cyclase activity. Other methods include GTPγS assays, inhibition of cAMP production assays and reporter gene assays (A Practical Approach Edited by G. Milligan. Oxford University Press (1999, supra).

Alternatively, the method can include detecting the induction of a reporter gene, which includes a CB receptor target-responsive regulatory element operatively linked to a nucleic acid encoding a detectable marker, e.g., luciferase.

In another embodiment, inhibitory agents of CB agonist-stimulated receptor expression are identified in a method wherein a cell is contacted with a test compound and the expression of CB receptor mRNA or protein in the cell is determined. The level of expression of CB receptor mRNA or protein in the presence of the test compound is compared to the level of expression of CB receptor mRNA or protein in the absence of the test compound. The test compound can then be identified as an inhibitor of CB receptor expression based on this comparison.

A cell which expresses a CB receptor can include recombinant cells expressing one or more CB receptors. A recombinant cell which expresses a CB receptor can be produced by transforming a host cell with one or more recombinant molecules, each comprising one or more nucleic acid molecules encoding a CB receptor operatively linked to an expression vector containing one or more transcription control sequences. An expression vector is a vector that is capable of transforming a host cell and of effecting expression of a specified nucleic acid molecule. The expression vector may be capable of replicating within the host cell or may integrate into one or more chromosomes of the host cell. Expression vectors can be either prokaryotic or eukaryotic, and are typically viruses or plasmids. Expression vectors useful in the present invention include any vectors that function (i.e., direct gene expression) in recombinant cells as described herein, including in bacterial, fungal, insect and mammalian cells.

Preferred recombinant molecules include any nucleic acid molecule which can express a CB receptor, or a biologically active portion thereof. The nucleic acid sequences and amino acid sequences of CB1\CB2 receptors from different animal species are known in the art. Swissprot and Emb1 numbers for the sequences for human, mouse, and rat are provided below. The invention will be equally applicable to new members of the cannabinoid receptor family as and when these are identified.

| | Swissprot | EMBL | | | | |
|---|---|---|---|---|---|---|
| Human CB1-R | p21554 | x54937 | x81120 | af107262 | u73304 | |
| Human CB1a-R | p21554 | x81121 | | | | |
| | splice variant | | | | | |
| Mouse CB1-R | p47746 | u17985 | u22948 | u40709 | af153345 | y18374 |
| Rat CB1-R | p20272 | x55812 | u40395 | | | |
| Human CB2-R | p34972 | x74328 | | | | |
| Mouse CB2-R | p47936 | x86405 | u21681 | x93168 | | |
| Rat CB2-R | Q9QZN9 | af176350 | | | | |

These database entries also identify published papers disclosing the cloning and sequencing of the various genes/proteins. For example, CB1R sequence is disclosed in Gerard C., Mollereau C., Vassart G., Parmentier M.; Nucleotide sequence of a human cannabinoid receptor cDNA. Nucleic Acids Res. 18:7142-7142(1990).

In another method, the method is a non-cell based method. In this assay, a CB receptor is contacted with a test compound in the presence of a CB receptor ligand and the ability of the test inhibitory agent to inhibit the binding of the CB receptor to the CB receptor ligand is determined.

Characterising if a CB Receptor Inhibitory Agent is a True Antagonist or an Inverse Agonist

The presently claimed method provides a means of determining if an identified inhibitory agent is a true antagonist or an inverse agonist. Determining if an identified inhibitory agent affects a CB receptor's intrinsic activity is difficult to measure and currently available methods do not allow the easy and accurate functional determination of an inhibitory agent. To overcome this problem, constitutively active CB receptors which have higher level of intrinsic activity were generated.

Constitutively Active CB Receptor

The present method includes the use of a constitutively active CB receptor which has an intrinsic activity greater than the wild-type CB receptor activity. The use of this consititutively active form of the CB receptor provides a means of accurately characterising the identified inhibitory agent as a true antagonist or an inverse agonist.

To generate such a consititutively active CB receptor, mutant CB receptors can be generated by standard techniques, such as site-directed mutagenesis and PCR-mediated mutagenesis. Commercially available kits can also be used such as the Quick change site-directed mutagenesis kit commercially available from Stratagene. The mutated CB receptors are then assayed to determine if there is an increase in intrinsic receptor activity. In one example, a cell is transformed with a nucleic acid molecule encoding the mutant CB receptor operatively linked to an expression vector containing one or more transcription control sequences. The intrinsic activity of the mutant CB receptor as compared to the activity of the wild type CB1 receptor is determined by detecting induction of a cellular second messenger such as cAMP, MAP kinase, or Ca²⁺. Assays that can be used to measure receptor mediated intracellular signalling are described above.

n one example, the CB1 receptor nucleic acid is mutated such that it encodes an alanine instead of an aspartic acid at position 3:49 (numbering system is that proposed by Ballesteros J A and Weinstein H (1995) Methods Neurosci 25, 366-428) and shows an increase in intrinsic activity compared to the intrinsic activity of the wild type CB1 receptor. In another example, the CB1 receptor nucleic acid is mutated such that it encodes an alanine instead of an aspartic acid at position 6:32 and shows an increase in intrinsic activity compared to the intrinsic activity of the wild type CB1 receptor. When compared to the wild type CB1, the CB1 is mutated to an alanine at position 213 and/or an alanine at position 338 of the human wild type CB1 (Swiss Prot P21554).

Based on the present disclosure, one skilled in the art would not just be able to easily generate other constitutively active CB1 receptors from other species, but also be easily able to generate other constitutively active cannabinoid receptors such as CB2 and CB1a receptors. This could be done by a process of identifying amino acids equivalent to those mutated as disclosed herein for the other CB receptors. This process is made easy by the numbering system proposed by Ballesteros, as this numbering system was designed such that it is possible to easily identify equivalent areas in all different GPCRs.

Assay Method

The constitutively active CB receptors provide a means of accurately characterizing an inhibitory agent. A number of different assay methods are provided below, however, these methods are not intended to be limiting.

The assay methods of the present invention can be performed in vitro e.g., using tissues, cells (e.g., HEK293 or CHO cells transiently expressing the wild type and mutant receptors) or using cell membrane preparations thereof. In vivo methods can also be used, for example, transgenic animals expressing a constitutively active CB receptor can be generated and these animals can be used to determine if an inhibitory agent acts as a true antagonist or inverse agonist. Methods for measuring the inhibitory agent's affect on CB receptor activity are described herein and are also applicable using tissues and cells isolated from the transgenic animal. Methods for generating transgenic animals are well known in the art.

Most conveniently, the method is performed in vitro. In one method, a test inhibitory agent is added to a recombinant cell which expresses a constitutively active CB receptor. The identity of the inhibitory agent is detected by determining if the inhibitory agent can inhibit the constitutive activity of the CB receptor. This can be done by determining if the inhibitory agent can inhibit second messenger induction such as adenylate cyclase, MAP kinase, or Ca²⁺. If the inhibitory agent can inhibit the constitutive activity of the CB receptor, the inhibitory agent is an inverse agonist.

In another method, a cell-based assay in which (i) a cell which expresses a constitutively active CB receptor is contacted with a test inhibitory agent, and (ii) a cell which expresses a wild-type CB receptor and which has been activated by a CB receptor agonist, is contacted with the test inhibitory agent. The intrinsic activity of the wild-type CB receptor is determined prior to addition of the agonist to the cell expressing the wild type CB receptor. The functional identity of the test inhibitory agent can be determined as follows.

If the inhibitory agent is a true antagonist, then it will inhibit the receptor activity of the agonist stimulated wild type CB receptor, but not affect the intrinsic activity of the receptor. It will also have no inhibitory affect on the constitutively active receptor's activity.

However, if the inhibitory agent is an inverse agonist it will inhibit the activity of the agonist activated wild-type CB receptor to levels below that of its intrinsic activity, and would inhibit the intrinsic activity of the constitutive CB receptor.

CB agonists that can be used to stimulate the wild-type CB receptor in the methods described above are well known in the art. For example, useful endogenous agonists of the CB1 receptor include anadamide and 2-arahidonylglycerol, and useful endogenous agonists of the CB2 receptor include anandamide and palmitoylethanolamide. In addition, CB1 and CB2 selective receptor agonists useful in the above method include CP-55,940, WIN55212-2, HU210, levonantradol, nabilone and methoananandamide.

The methods described above, can, instead of being performed using a whole cell, also be performed using a membrane preparation of these cells. Membrane preparations can be made by any method known in the art. For example, as described in Signal Transduction: A Practical Approach Edited by G. Milligan. Oxford University Press (1999))

Therapeutics

True CB receptor antagonists and inverse agonists of the CB receptors can be used as therapeutic agents useful in the treatment or prevention of CB associated diseases. For example, a true antagonist or inverse agonist can be used for the treatment of obesity, psychiatric disorders such as psychotic disorders, anxiety, anxio-deoressive disorders, depression, cognitive neurological disorders such as dementia, multiple sclerosis, Raynaud's syndrome, Parkinson's disease, Huntington's chorea and Alzheimer's disease. A true antagonist or inverse agonist of the CB receptor are also potentially useful for the treatment of immune cardiovascular, reproductive and endocrine disorders, and also diseases related to the respiratory and gastronintestinal systems.

The true antagonist or inverse agonist can be administered alone or in a mixture, in the presence of a pharmaceutically acceptable excipient or carrier. The excipient or carrier is selected on the basis of the mode and route of administration. The appropriate unit forms of administration include oral forms such as tablets, gelatin capsules, powders, granules and solutions or suspensions and can be administered orally, subcutaneously, intramuscularly, intravenously, transdermally, or locally.

The identified true antagonist or inverse agonist can be combined with other therapeutic agents which are useful in the treatment of CB associated disorders such as obesity.

Pharmaceutical compositions comprising a true antagonist or inverse agonist are generally formulated in dosage units. The dosage unit contains from 0.5 to 1000 mg, advantageously from 1 to 500 mg and preferably from 2 to 200 mg of a CB receptor true antagonist or inverse agonist per dosage unit for daily administration.

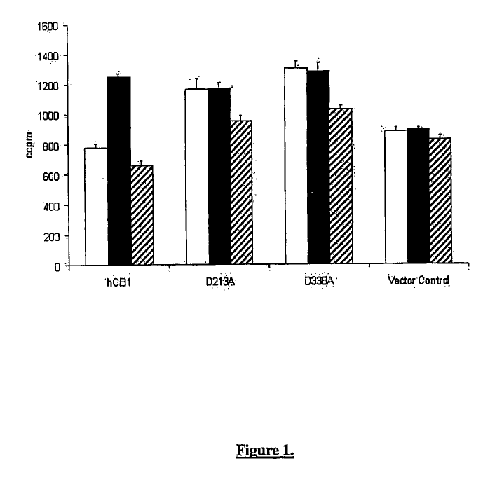

The invention will now be further illustrated by the following non-limiting examples and FIG. 1 which shows GTPγS activity of membranes prepared from HEK293 cells transiently transfected with plasmids containing human CB1 cDNA, or either of two mutants D213A or D338A or vector control. Membranes were incubated in the absence of any compound (clear—□); 10 μM CP55940 (solid—▪); or, 10 μM SR141716 (striped) and the respective GTPγS activity determined.

### EXAMPLES

### Example 1

Point mutations were introduced into the human CB1 receptor nucleic acid sequence using the Quick change site-directed mutagenesis kit (commercially available from Stratagene; product #200518) according to the manufacturers recommendations. Oligonucleotides containing single nucleotide mismatches with the wild type CB receptor sequences were designed and used together with the Stratagene kit to introduce single nucleotide mutations in the cDNAs. Specifically, codons in the oligonucleotides encoding aspartic acid, GAC at position 3:49 (numbering system is that proposed by Ballesteros J A and Weinstein H (1995) Methods Neurosci 25, 366-428) and GAT at position 6:32, were altered to the alanine encoding codons GCC and GCT respectively.

SEQ ID NO: 1 represents the amino acid sequence of hCB1-D213A, D388A double mutant. SEQ ID NO: 2 represents the encoding nucleotide sequence of the hCB1 double constitutive mutant. SEQ ID NO:3 represents the encoding nucleotide sequence of the hCB1 D213A constitutive mutant. SEQ ID NO:4 represents the encoding nucleotide sequence of the hCB1 D388A constitutive mutant.

Mutant cDNA's were then transiently transfected into HEK293 cells. Membrane preparations were prepared by resuspending receptor expressing cells in ice cold TE buffer (10 mM Tris-HCl, 0.1 mM EDTA pH7.5) and leaving on ice for 5 minutes before pelleting the insoluble material by centrifugation at 1000 g. This process was repeated twice before resuspending the pellet in an appropriate volume of TE and storing at −80° C. The activity of the mutant receptor was determined using a GTPS binding assay as follows: 10 μg of membranes diluted in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. To determine the level of non-specific binding contol reactions were performed in the presence of 10 μM GTPγS.

Functional activity of compounds at wild type and mutant receptors, either in the presence or absence of agonist, were determined as follows: Non-specific binding was subtracted from all values determined. Maximum activity was that determined in the presence or absence of an agonist but in the absence of any antagonist/inverse agonist following subtraction of the value determined for non-specific activity. The effect of compounds at various concentrations was plotted according to the equation:
y=A+((B−A)/1+((C/x)\hat{ }D)))
and IC₅₀ estimated where
A is the bottom plateau of the curve i.e. the final minimum y value
B is the top of the plateau of the curve i.e. the final maximum y value
C is the x value at the middle of the curve. This represents the log EC50 value when A+B=100
D is the slope factor.
x is the original known x values.
Y is the original known y values.
\hat{ } is to the power of.

1. A method for identifying an inverse agonist of a CB receptor, the method comprising:
measuring the activity of a constitutively active CB receptor;
contacting a CB receptor test inhibitory agent with the constitutively active CB receptor; and
measuring the activity of the constitutively active CB receptor following contact with the inhibitory agent, wherein a decrease in the activity in the constitutively active CB receptor, compared to the activity of the constitutively active CB receptor in the absence of the inhibitory agent, indicates that the agent is an inverse agonist.

2. A method for determining if a CB receptor inhibitory agent is an inverse agonist or a true antagonist of a CB receptor, the method comprising:
providing a test CB receptor inhibitory agent;
contacting the agent with a wild-type CB receptor in the presence of a CB receptor agonist; contacting the agent with a constitutively active CB receptor;
measuring the activity of the wild-type CB receptor and the constitutively active CB receptor, wherein:
(i) a decrease in the activity in both the wild-type CB receptor and the constitutively active CB receptor indicates that the agent is an inverse agonist, or
(ii) a decrease in the activity in the wild-type CB receptor, but not of the activity of the constitutively active CB receptor, indicates that the compound is a true antagonist.

3. A method for identifying an inverse agonist of a CB receptor, the method comprising:
measuring the activity of a constitutively active CB receptor expressed in a cell;
contacting a CB receptor test inhibitory agent with the cell expressing the constitutively active CB receptor; and
measuring the activity of the constitutively active CB receptor following contact with the inhibitory agent, wherein a decrease in the activity in the constitutively active CB receptor compared to the activity of the constitutively active CB receptor in the absence of the inhibitory agent indicates that the agent is an inverse agonist.

4. A method for determining if a CB receptor inhibitory agent is an inverse agonist or a true antagonist of a CB receptor, the method comprising:
providing a test CB receptor inhibitory agent;
contacting the agent with a cell expressing a wild-type CB receptor in the presence of a CB agonist;
contacting the agent with a cell expressing a constitutively active CB receptor;
measuring the activity of the wild-type CB receptor and the constitutively active CB receptor, wherein
(i) a decrease in the activity in both the wild-type CB receptor and the constitutively active CB receptor indicates that the agent is an inverse agonist; or
(ii) a decrease in the activity in the wild-type CB receptor in the presence of a CB receptor agonist, but not the activity of the constitutively active CB receptor, indicates that the compound is a true antagonist

5. The method of any of claims 1, 2, 3, or 4 wherein the constitutively active CB receptor is a CB1 receptor, or a variant thereof, CB2 receptor, or a variant thereof.

6. The method of claims 2 or 4, wherein the wild-type CB receptor is a CB1 receptor, or a variant thereof, CB2 receptor, or a variant thereof.

7. The method of claim 5, wherein the constitutively active CB1 receptor is a human CB1 receptor comprising an alanine at position 213.

8. The method of claim 5, wherein the constitutively active CB1 receptor is a human CB1 receptor comprising an alanine at position 338.

9. The method according to claims 3 or 4, wherein the cell is a mammalian cell, an insect cell, or a yeast cell.

10. The method according to claims 2 or 4, wherein the CB agonist is CP55940 or HU210.

11. A true antagonist or an inverse agonist identified by the method of any one of claims 1-4.

12. A pharmaceutical formulation comprising a true antagonist or an inverse agonist as identified by the method of any one of claims 1-4, and a pharmaceutically acceptable adjuvant, diluent or carrier.

13. (canceled)

14. (canceled)

15. A method of treating a CB associated disorder comprising administering a pharmacologically effective amount or the true antagonist or inverse agonist as identified by the method of any one of claims 1-4 to a patient in need thereof.

16. The method of claim 15, wherein the disorder is obesity.

17. A constitutively active CB receptor.

18. The receptor of claim 17, wherein the receptor is a human CB1b receptor.

19. The method of claim 18, wherein the receptor comprises an alanine at position 213 of the human wild type CB1b receptor.

20. The method of claim 18, wherein the receptor comprises an alanine at position 338 of the human wild type CB1b receptor.

21. An isolated nucleic acid sequence comprising a nucleotide sequence that encodes a variant cannabinoid receptor protein wherein one or both of the amino acids located at positions 3:49 and 6:32 have been substituted for by another amino acid so as to create a constitutive vaiant form of the cannabinoid receptor.

22. The isolated nucleic acid according to claim 21, wherein the cannabinoid receptor protein is a human CB1 receptor.

23. The isolated nucleic acid according to claim 22, wherein one or both of the amino acids at positions 213 and 338 of the CB1 receptor protein is an alanine residue.

24. The isolated nucleic acid according to claim 23, wherein the nucleic acid comprises the sequence according to any of SEQ ID NOs: 2, 3 or 4.

25. A vector comprising the nucleic acid molecule as claimed in claim 21.

26. A cell or cell line transformed with the vector of claim 25.

27. The cell or cell line according to claim 26, which is a bacterial, yeast, insect or mammalian cell or cell line.

28. An isolated cannabinoid receptor polypeptide, wherein one or both of the natural amino acids at positions 3:49 and 6:32 of the receptor polypeptide have been substituted for another amino acid.

29. The isolated polypeptide of claim 28, wherein the cannabinoid receptor is CB1.

30. The isolated polypeptide of claim 29, wherein one or both of the amino acids at positions 3:49 and 6:32 of CB1 is an alanine residue.

31. An isolated human cannabinoid 1 receptor polypeptide comprising the sequence according to SEQ ID NO: 1, with the exception that one or both of the amino acids at position 213 or 338 may be an aspartic acid residue.


SEQUENCE LISTING (4 sequences)

SEQ ID NO: 1 - protein, 472 aa, Homo sapiens
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIARYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMAIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 2 - DNA, 1419 bp, Homo sapiens
atgaagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgccaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggacattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctgtga

SEQ ID NO: 3 - DNA, 1419 bp, Homo sapiens
atgaagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggccattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctgtga

SEQ ID NO: 4 - DNA, 1419 bp, Homo sapiens
atgaagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgccaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggccattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctgtga